(19) United States
(12) Patent Application Publication
(10) Pub. No.: US 2001/0006963 A1
(43) Pub. Date: Jul. 5, 2001

(54) ONCE-A-MONTH INJECTION AS A DEPOT CONTRACEPTIVE AND FOR HORMONE REPLACEMENT THERAPY FOR PERIMENOPAUSAL AND PREMENOPAUSAL WOMEN

(76) Inventors: URSULA LACHNIT-FIXSON, BERLIN (DE); SEMIRAMIS AYDINLIK, BERLIN (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/348,232

(22) Filed: Jul. 6, 1999

Related U.S. Application Data
(63) Continuation of application No. 08/913,306, filed on Jan. 30, 1998, now abandoned.

(30) Foreign Application Priority Data
Mar. 16, 1995 (DE) 195 10 861.2

(51) Int. Cl. A61K 31/70; A61K 31/56

(52) U.S. Cl. 514/171

(57) ABSTRACT

This invention describes the use of a once-a-month injection (once-a-month injectable) that contains an estrogenic and gestagenic component as an active ingredient for the production of a pharmaceutical agent for contraception and simultaneous hormone replacement therapy for perimenopausal and premenopausal women.

With this pharmaceutical agent, a more reliable contraceptive effect with a “natural” estrogen (without ethinylestradiol) with accompanying active therapy of the beginning estrogen-loss symptoms, as well as prevention of osteoporosis, is achieved.

This invention relates to the use of a once-a-month injection (once-a-month injectable composition) that contains an estrogenic and gestagenic component as an active ingredient for the production of a pharmaceutical agent for contraception and simultaneous hormone replacement therapy for perimenopausal and premenopausal women.

A once-a-month injection as defined by this invention means a hormone preparation that is injected in women of child-bearing age once a month for contraception. In this hormone preparation, a gestagenic as well as an estrogenic component are contained as active substances, each with a sufficiently long action to achieve a contraceptive effect for a one-month period.

So-called progestogen-only injectables are also available, which ensure longer-lasting contraceptive protection, but with poor cycle control.

The best known representatives of such a once-a-month injection are Cyclofem (HRP 112; Cycloprovera) and Mesigyna (HRP 102). The former contains 25 mg of medroxyprogesterone acetate as a gestagenic component and 5 mg of estradiol cyprionate as an estrogenic component in a microcrystal suspension, while the latter contains 50 mg of norethisterone enanthate as a gestagenic component and 5 mg of estradiol valerate as an estrogenic component in an oily solution. Although the women that select these methods of prevention have to visit a family planning center or a physician each month to have the injection administered, these once-a-month injections are readily used for contraception. Vaginal bleeding occurs like menstruation more or less regularly and predictably, which is of tremendous importance for ensuring broad acceptance of these reliable methods in many cultural groups. (Biennial Report 1988-1989, Research in Human Reproduction, WHO).

A method for hormonal treatment of menopausal, including perimenopausal and postmenopausal, disorders is already described in U.S. Pat. No. 4,826,831, which especially relates to a treatment during continuous use of a gestagen in connection with an estrogen. In this case, the estrogen can also be used continuously or else cyclically, i.e., with pauses in intake.

A considerable number of estrogens and gestagens are known that are suitable for the purpose of this invention, i.a., even norethisterone acetate and medroxyprogesterone acetate as gestagens as well as estradiol valerate. In principle, all estrogens and gestagens that also are conceivable in oral contraceptives should be usable. Adequate contraceptive protection is not expressly provided by the described treatments.

In addition, norethisterone acetate/estradiol valerate is disclosed as a possible combination. The quantities of gestagen and estrogen that are used are low: the gestagen corresponds to a daily quantity that corresponds to a quantity of 0.025 mg to 0.075 mg of levonorgestrel, and the estrogen corresponds to a daily quantity that corresponds to a quantity of 0.5 to 2.0 mg of estradiol.

In the case of oral administration of active ingredients, the quantity of estradiol valerate that is to be administered daily is in a range of 0.5 mg to 2.0 mg; a daily quantity of 0.1 mg to 1.0 mg is indicated for norethisterone acetate, and a quantity of from 1.0 mg to 15.0 mg is indicated for medroxyprogesterone acetate. U.S. Pat. No. 4,826,831 also comprises the non-oral administration of the indicated active ingredients using implants or by intramuscular injection. The required “daily” dosages are then somewhat lower than in the case of oral administration because of the direct transition of the active ingredients into the blood stream.

20 mg to 100 mg of estradiol valerate is indicated for an estrogen implant. A gestagen depot formulation for 3 months is to contain 50 to 500 mg of medroxyprogesterone acetate or 20 to 400 mg of norethisterone enanthate.

This patent also relates to pharmaceutical compositions for implementing this method.

With this method, the symptoms that are associated with the natural removal of estrogen, which begins as early as in premenopause, such as, for example, hot flashes, dryness of the vagina, the risk of osteoporosis, and the increasing risk in women over age 60 of suffering myocardial infarction (cardiovascular complications), are effectively counteracted, whereby based on the gestagenic components, however, induction of bleeding is suppressed, but the risk of endometrial carcinomas forming is not increased.

U.S. Pat. No. 4,826,831 expressly mentioned that in the case of oral administration, this is not a method of contraception.

In terms of this invention, perimenopause and premenopause are to be defined with their conventional meanings, as is indicated in “The Controversial Climacteric,” P. A. van Keep et al., Ed., MTP Press (1981) on page 9.

The object of this invention is to make available a depot pharmaceutical agent that is suitable for women in perimenopause and premenopause equally for still necessary contraception and for hormonal substitution therapy (Hormone Replacement Therapy= HRT) that is advisable already in this phase of life.

It has now been found that such a pharmaceutical agent for premenopausal and perimenopausal women can be produced, surprisingly enough, based on a once-a-month injection. While bleeding of the women under treatment is to be especially avoided by the methods that are described in U.S. Pat. No. 4,826,831, monthly bleeding occurs in perimenopausal or premenopausal women after administration of the once-a-month injection according to the invention at the end of the treatment period; thus no amenorrhea is induced.

Once-a-month injections in terms of this invention are to be defined both as the already initially mentioned products and all other conceivable combinations of a natural estrogen with a gestagen. In this case, the two active components have to be present in a depot formulation, whereby the depot effect is achieved either by the special type of galenical formulation (for example, a microcrystal suspension) or else, more likely, by the chemical structure of the gestagen and/or estrogen (for example, by the esterification of free hydroxy groups).

In principle, all natural estrogens and all gestagens are considered that are suitable for use in oral contraceptives, and they can be converted in the above-mentioned way into a galenical formulation or a chemical form can be derived that produces a depot effect, and the dosage form that is to be administered intramuscularly can be produced. As estrogens, primarily 17β-estradiol, estradiol-3-benzoate, estradiol-17-valerate, -cypionate, -undecylate, -enanthate and/or other estradiol esters are suitable here (U.S. Pat. Nos. 2,611,773, 2,990,414, 2,054,271, 2,225,419 and 2,156,599).

The gestagenic component is preferably selected from the groups of compounds norethisterone acetate, norethisterone enanthate, medroxyprogesterone acetate, and cyproterone acetate.

Selected estrogens or gestagens that are to be used according to this invention are listed in the following tables 1A and 2A with indication of the preferred quantity range. The especially preferred quantities of the respective estrogen or gestagen, which are to be contained in a once-a-month injection according to this invention, are described in Tables 1B and 2B.

TABLE 1A

| | Minimum Dosage | Maximum Dosage |
|---|---|---|
| | (mg) | (mg) |
| 17β-Estradiol | 4 | 10 |
| Estradiol valerate | 4 | 10 |
| Estradiol cipionate | 4 | 10 |

TABLE 1B

| | Dosage |
|---|---|
| | (mg) |
| Estradiol 17B | 5 |
| Estradiol valerate | 5 |
| Estradiol cipionate | 5 |

TABLE 2A

| | Minimum Dosage | Maximum Dosage |
|---|---|---|
| | (mg) | (mg) |
| Norethisterone | 20 | 60 |
| acetate | | |
| Norethisterone | 20 | 60 |
| enanthate | | |
| Medroxyprogesterone | 12.5 | 30 |
| acetate | | |
| Cyproterone acetate | 40 | 100 |

TABLE 2B

| | Dosage |
|---|---|
| | (mg) |
| Norethisterone acetate | 50 |
| Norethisterone enanthate | 50 |
| Medroxyprogesterone acetate | 25 |
| Cyproterone acetate | 50 |

According to the invention, the combination of medroxyprogesterone acetate/estradiol cypionate is preferred for the production of a once-a-month injection for perimenopausal and premenopausal contraception; the norethisterone enanthate/estradiol valerate combination is especially preferred.

As special advantages of the once-a-month injection that is produced according to the invention, which can be used on premenopausal women, surprisingly enough, both for contraception and for hormone replacement therapy, the following can primarily be mentioned:

a more reliable contraceptive effect without ethinylestradiol with “natural” estrogen;

accompanying active therapy of the beginning estrogen-loss symptoms (elimination of menopausal symptoms);

simultaneous optimum osteoporosis prevention;

outstanding tolerance and cycle control;

virtually no change in blood pressure;

very low, almost nonexistent undesirable side-effects and influences on metabolic and hemostatic parameters;

avoidance of burdening the liver, as well as of gastrointestinal disorders based on the parenteral method of application;

preserving the advantageous side effects of oral contraceptives (protection from ovarial and endometrial carcinomas and pelvic inflammatory disease [PID]).

All these properties were documented by a 3-year clinical study with Mesigyna, whereby 17- to 35-year-old women participated in the group of patients.

The especially preferred embodiment based on the gestagen/estrogen combination of norethisterone enanthate/estradiol valerate offers not only a prophylactic effect with respect to osteoporosis, but even a bone build-up effect caused by norethisterone enanthate is observed in this combination owing to the bone-degradation-inhibiting action of the estrogen.

### FORMULATION EXAMPLES

### Mesygina

An ampoule contains

50 mg of norethisterone enanthate+ 5 mg of estradiol valerate

in 1 ml of castor oil/benzyl benzoate 6/4 (V/V).

### Formulation with Cyproterone Acetate

An ampoule contains

50 mg of cyproterone acetate+ 5 mg of estradiol valerate

in 1 ml of castor oil/benzylbenzoate 6/4 (V/V).

1. Use of a once-a-month injection (once-a-month injectable) that contains estrogenic and gestagenic components as an active ingredient for the production of a pharmaceutical agent for contraception and simultaneous hormone replacement therapy for perimenopausal and premenopausal women.

2. Use according to claim 1, characterized in that the estrogenic component is selected from the group of compounds 17β-estradiol, estradiol-3-benzoate, estradiol-17-valerate, -cypionate, -undecylate, -enanthate as well as other estradiol esters.

3. Use according to claim 1, wherein the gestagenic component is selected from the group of compounds norethisterone acetate, norethisterone enanthate, medroxyprogesterone acetate, cyproterone acetate.

4. Use of the combinations of
norethisterone enanthate/estradiol valerate,
medroxyprogesterone acetate/estradiol cypionate according to claims 2 and 3.
